(19) United States
(12) Patent Application Publication
(10) Pub. No.: US 2010/0286034 A1
(43) Pub. Date: Nov. 11, 2010

(54) USES FOR AQUEOUS STREAMS CONTAINING PROTEINS

(76) Inventors: Pieter Marinus Broecke Van Den, Didam (NL); Luppo Edens, Rotterdam (NL); Andre Leonardus Roos, Delft (NL)

Correspondence Address: NIXON & VANDERHYE, PC, 901 NORTH GLEBE ROAD, 11TH FLOOR, ARLINGTON, VA, 22203, US

(21) Appl. No.: 12/811,051

(22) PCT Filed: Dec. 10, 2008
(86) PCT No.: PCT/EP08/67168

§ 371 Date: Jun. 28, 2010

(30) Foreign Application Priority Data
Dec. 28, 2007 (EP) 07124127.7

(51) Int. Cl. A61K 38/06; C12P 21/06; A61P 3/10; A61P 5/50; A61P 3/04; A23J 1/00

(52) U.S. Cl. 514/4.8; 435/68.1; 514/6.7; 514/6.9; 426/656

(57) ABSTRACT

The present invention relates to a protein hydrolysate comprising free amino acids and peptides whereby the weight ratio of free amino acids to peptides is about 1:1 and wherein at least about 50 molar % of the peptides has a molecular weight of 400 Da or less. This composition may be rich in one or more branched-chain amino acid-(BCAA-) and or glutamine-containing di- or tripeptides. Also, the invention relates to the use of a water soluble protein-containing aqueous fraction obtained from a wet-milling process or the protein hydrolysate in: the manufacture of a medicament for the treatment or prevention of a condition associated with inappropriate blood sugar metabolism; aiding recovery and/or endurance during or after exercise; stimulating the generation of lean body mass; infant nutrition; or the preparation of a food or feed composition or a food or feed supplement.

### FIELD OF THE INVENTION

The present invention relates to protein-containing compositions and to their use in: the treatment of conditions associated with inappropriate blood sugar metabolism; aiding recovery and/or endurance during or after exercise; stimulating the generation of lean body mass; and the preparation of a food or feed composition or a food or feed supplement. The invention also relates to food and food compositions or supplements prepared using the protein-containing compositions.

### BACKGROUND OF THE INVENTION

Hydrolysed proteins from a variety of sources are used in the food and food supplement industries.

For instance, they are commonly employed as a component in dehydrated soups, as flavourings and in other processed foodstuffs to obtain e.g. food flavourings after a Maillard reaction. In food applications, in situ hydrolysed proteins play an important role in the development of flavours in fermented products. In the latter products the microbial starter cultures used usually excrete proteolytic enzymes responsible for hydrolysis of the raw material into peptides and free amino acids. Metabolic transformation of these free amino acids leads to potent flavour compounds and volatile characteristics for e.g. fermented dairy products such as cheese or yogurts, various meat products, beers and wines.

Hydrolysed proteins rich in peptides and with relatively low levels of free amino acids are frequently used in infant nutrition. They also find medical use as dietary supplements for patients suffering from a variety of diseases and metabolic disorders. Newer developments include their use in products for consumers with non-medical needs such as athletes, for example in sports drinks to aid recovery and endurance, and individuals on a slimming diet. Protein hydrolysates represent a rich source of amino acids to stimulate protein synthesis and thus have a positive influence on raising or maintaining muscle mass. Protein hydrolysates may thus be used by all people who need to maintain or increase their muscle mass, for example overweight people during a calorie restricted diet, children, women, the elderly and those who often skip meals. An increased protein intake combined with resistance training can stimulate muscle growth in people who are on a calorie restricted diet and increased muscle mass that burns more calories may help to improve body shape and make it easier to maintain body weight. Furthermore protein hydrolysates are being used in personal care applications.

Even more recently, protein hydrolysates have found application in the treatment of certain medical conditions, for example in the treatment of type 2 diabetes mellitus (T2DM), pre-diabetes, metabolic syndrome, obesity and to prevent long-term complications in subjects with such disorders. The basis for this application is the observation that the dietary intake of protein hydrolysates stimulates insulin secretion.

Protein hydrolysates rich in free amino acids are typically prepared either by subjecting a protein source to harsh chemical conditions or by enzymatic hydrolysis. Both the chemical and the enzymatic route aim to release high levels of amino acids from the protein source with maximum efficiency and lowest cost. For that reason, cheaply available protein sources like soy meal and wheat gluten are popular substrates for preparing hydrolysates. To liberate as many amino acids as possible, the enzymatic route employs either complex mixtures of several endo- and exoproteases or combines endoproteases with a single but broad spectrum exoprotease. Typically, the aim is to obtain a high degree of hydrolysis and an end product that contains a large variety of free amino acids.

WO98/14599 refers to certain polypeptides obtained from Aspergillus oryzae and to hydrolysates prepared with these polypeptides in combination with (specific or unspecific) endopeptidases and (specific or unspecific) exopeptidases. WO98/14599 mentions hydrolysates that have an increased content of Leu, Gly, Ala and/or Pro, such as 1.1 times greater but uses for such hydrolysates are not mentioned.

Voigt et al. (Food Chemistry 51 (1994) pp. 7-14) describe the production of the cocoa-specific aroma precursors by in vitro proteolysis of seed proteins. Cocoa-specific aroma precursors can only be obtained by specific hydrolysis of only one substrate, which is cocoa vicillin-class globulin proteins.

WO02/032232 describes a protein hydrolysate in which a single desired amino acid is enriched in the hydrolysate at least 2.5 times higher than would have been obtained by acid hydrolysis of the same protein-containing substrate.

All of the methods described above to prepare the various hydrolysates require significant quantities of enzymes that are relatively expensive. Furthermore, the hydrolysates used for modulating physiological aspects, require specific amino acid compositions to be optimally active. Accordingly, there is a need for a suitable protein source which may be used as is or with minimal enzyme treatment in the applications mentioned above.

### SUMMARY OF THE INVENTION

In recent years, the benefits of protein hydrolysates in a number of applications, such as stimulating insulin production in pre-diabetic persons, generating lean body mass, modulating flavour in food/feed preparation and in aiding recovery and endurance, for example during or after physical exercise, have become clear.

The literature available indicates that branched chain amino acids and more specifically the amino acid leucine play an important role in these phenomena. Branched amino acids are also known to play an important role in the treatment of various diseases including liver cirrhosis, sepsis, trauma and chronic fatigue syndrome. By the term “branched chain amino acid” (BCAA), the amino acids leucine, isoleucine and valine in particular are meant. Furthermore the literature available emphasizes the central metabolic role of the amino acid glutamine. Glutamine is known to improve physical endurance and recovery rate following high intensity exercise, probably via a stimulation of a whole body glucose utilization and maintenance of circulating glucose levels. Glutamine has also been mentioned in connection with enhanced immune function, a compromised gut, neurological activity and clinical nutrition in general.

We have now shown that supernatants of an aqueous stream isolated in the corn wet milling process, in particular the supernatant of corn gluten meal, is an excellent source of water-soluble, BCAA such as leucine-rich peptides. Additionally, such aqueous stream, for example the supernatant of corn gluten meal, is an excellent source of water-soluble, glutamine-rich peptides. As a free amino acid, glutamine is not stable so that supply in the form of a readily assimilable peptide is advantageous.

Prior to the present invention such products were economically difficult because glutamine- and proline-rich cereal derived proteins are particularly hard to hydrolyze leading to excessive enzyme costs.

However, evaporation, optionally followed by spray-drying of the supernatants may lead to a highly cost-effective concentrate, a paste or a dry powder derived from the wet-milling stream, for example from the supernatant of corn gluten meal.

Moreover, the dietary characteristics of such a product may further be enhanced by subjecting it to an incubation with relatively low concentrations of proteolytic enzymes. Such an incubation with selected proteolytic enzymes may result in a product in which, aside from the free amino acids present, up to 80% of all peptides are present in the form of readily absorbable di- or tripeptides rich in BCAA's and notably leucine as well as glutamine. Of the hydrolysates widely available for industrial application, only whey protein hydrolysates exhibit a composition as favourable as the corn gluten meal supernatant described herein. However, the corn derived product is significantly cheaper.

The invention thus relates to uses of a water soluble protein-containing aqueous fraction obtained from a wet-milling process. Such material will typically be the supernatant (i.e. soluble material-containing fraction) of an aqueous stream generated in a wet-milling process which supernatant comprises water soluble protein. A preferred example of such material is the supernatant from gluten meal, preferably corn gluten meal.

The material may be subjected to enzymatic hydrolysis. The invention thus also concerns a water-soluble composition comprising free amino acids and enzymatically hydrolyzed proteinaceous material yielding peptides whereby the weight ratio of free amino acids to peptides is between 3:1 and 1:3, preferably between 2:1 and 1:2, more preferably between 1.5:1 and 1:1.5, and wherein at least about 50 molar % of the peptides has a molecular weight of 400 Da or less. Preferably this composition is rich in branched chain amino acid (BCAA-) and/or glutamine. Preferably the peptides are rich in di- or tripeptides. Advantageously this composition is obtainable by the enzymatic protein hydrolysis of a water soluble protein fraction from a corn wet-milling process. So preferably this composition is a hydrolysate which is rich in one or more BCAA-, such as leucine-, containing and/or glutamine-containing di- and/or tri-peptides.

In the hydrolysate or water-soluble composition of the invention at least about 20%, preferably at least 20%, more preferably at least about 25%, still more preferably at least 25%, even more preferably at least about 30% or most preferably at least 30% of the amino acids present in the peptides and free amino acids is a BCAA and/or glutamine whereby BCAA is leucine and/or isoleucine and/or valine.

In the hydrolysate or water-soluble composition of the invention preferably at least 50 molar %, more preferably at least about 60 molar %, still more preferably at least 60 molar %, even still more preferably at least about 70 molar %, or most preferably at least 70 molar % of the peptides have a molecular weight of 400 Da or less.

Such a protein hydrolysate may be prepared by a method which comprises contacting of a water soluble protein-containing aqueous fraction obtained from a wet-milling process a protein substrate with:
a) an endoprotease; and
b) an exoprotease preferably a tripeptidase, more preferably a tripeptidyl aminopeptidase(TPAP).

The invention provides a hydrolysate obtainable by such a method. Especially by the use of an endoprotease and a tripeptidyl peptidase the amount of free amino acids (in wt %) will not change during hydrolysis. Also the amount of peptide bound amino acids will remain almost constant before and after hydrolysis.

The water soluble protein-containing aqueous fraction obtained from a wet-milling process may be used as is or in the form of a hydrolysate. The aqueous fraction or hydrolysate may be used in a concentrated form.

According to the invention, there is thus provided a water soluble protein-containing aqueous fraction from a wet-milling process or a hydrolysate thereof, so a water-soluble composition comprising free amino acids and peptides whereby the weight ratio of free amino acids to peptides is between 3:1 and 1:3, preferably between 2:1 and 1:2, more preferably between 1.5:1 and 1:1.5, and wherein, of the hydrolysate, at least about 50 molar % of the peptides has a molecular weight of 400 Da or less. Moreover the use of such a protein-containing aqueous fraction or its hydrolysate is provided in:
manufacturing a medicament for the treatment or prevention of a condition associated with inappropriate blood sugar metabolism;
aiding recovery and/or endurance during or after exercise;
stimulating the generation of lean body mass;
reducing diet-induced obesity;
improving cholesterol metabolism;
overcoming liver failure and cirrhosis;
preparing infant nutrition;
preparing a food or feed composition or a food or feed supplement; or
modulating the taste or aroma of fermented or heated food products.

Conditions which are associated with inappropriate blood sugar metabolism include diabetes, pre-diabetes, impaired glucose tolerance, metabolic syndrome and obesity.

The resulting food or feed composition or food or feed supplement may have improved properties owing to use of the water soluble protein-containing aqueous fraction obtained from a wet-milling process.

The properties of the resulting food or feed composition or food or feed supplement may be such that it will be useful in the treatment or prevention of a condition associated with inappropriate blood sugar metabolism; in aiding recovery and/or endurance during or after exercise; or in stimulating the generation of lean body mass; or in infant nutrition or to improve overall body line aesthetics, especially in combination with exercise. Furthermore, these improved properties of the resulting food or feed composition or food or feed supplement include beneficial effects on, for example, aroma and taste when the water soluble protein-containing aqueous fraction or hydrolysate of the invention is used as an ingredient in fermented or heated food or feed products (i.e. is used in the preparation of such food or feed products).

The invention also provides a water soluble protein-containing aqueous fraction obtained from a wet-milling process or a hydrolysate of the invention for use in the treatment or prevention of a condition associated with inappropriate blood sugar metabolism. Further, the invention provides a method for the treatment or prevention of a condition associated with inappropriate blood sugar metabolism, which method comprises administering to an individual in need thereof a therapeutically effective amount of a water soluble protein-containing aqueous fraction obtained from a wet-milling process or a protein hydrolysate of the invention.

Further provided by the invention is a method for the preparation of a food or feed composition which method comprises use of a water soluble protein-containing aqueous fraction obtained from a wet-milling process or a hydrolysate of the invention during the preparation of a said food or feed stuff.

Such a method may be used to modulate the organoleptic aspects of the resulting food or feed product.

The invention also provides a food or feed composition or a food or feed supplement prepared using a soluble fraction of a protein-containing material obtained from a wet-milling process or a hydrolysate of the invention. Such a food or feed composition or a food or feed supplement may be a fermented composition.

### BRIEF DESCRIPTION OF THE DRAWING

FIG. 1 shows the peptide size distribution of corn gluten supernatant after treatment with different proteases. “as such”: peptide size distribution of corn gluten supernatant without enzymatic treatment. “subtilisin”: after treatment with Protex; “PSE”: after treatment with proline-specific endoprotease; “TPAP”: after treatment with tripeptidyl aminopeptidase; “PSE+TPAP”: after treatment with both proline-specific endoprotease and tripeptidyl aminopeptidase.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The invention relates to uses for a water soluble protein-containing aqueous fraction obtained from a wet-milling process.

The water soluble protein-containing aqueous fraction obtained from a wet-milling process described herein, may be subjected to enzymatic hydrolysis. The resulting hydrolysate is enriched in respect of BCAA-, i.e. one or more of leucine, isoleucine and valine, and glutamine-containing di- or tripeptides. The invention thus relates to such a protein-containing fraction and its hydrolysate which is a water-soluble composition comprising free amino acids and peptides whereby the weight ratio of free amino acids to peptides is between 3:1 and 1:3, preferably between 2:1 and 1:2, more preferably between 1.5:1 and 1:1.5. Of the hydrolysate at least about 50 molar % of the peptides has a molecular weight of 400 Da or less whereby the peptides are rich in di- or tripeptides. In the hydrolysate or water-soluble composition of the invention at least about 20%, preferably at least 20%, more preferably at least about 25%, still more preferably at least 25%, even more preferably at least about 30% or most preferably at least 30% of the amino acids present in the peptides and free amino acids is a BCAA and/or glutamine whereby BCAA is leucine and/or isoleucine and/or valine. This composition has advantageously a vegetable origin and is preferably the hydrolysate of a vegetable protein or vegetable protein fraction such as a fraction obtained from a protein wet-milling process, preferably a corn wet-milling process. So the composition has advantageously a corn origin and is preferably the hydrolysate of a corn protein or corn protein fraction such as a fraction obtained from a corn wet-milling process.

In the hydrolysate of the water-soluble composition of the invention preferably at least 50 molar %, more preferably at least about 60 molar %, still more preferably at least 60 molar %, even still more preferably at least about 70 molar %, or most preferably at least 70 molar % of the peptides have a molecular weight of 400 Da or less.

The water soluble protein-containing aqueous fraction to be used according to the invention can be obtained from any grain that may be subjected to wet milling, but the grain is preferably corn (maize).

Wet-milling is generally achieved by a combination of chemical and mechanical means. Wet-milling grain involves soaking the grain. In the corn wet-milling process the soaking of the grain is termed “steeping”. The steeping process of corn, generally, includes the addition of sulphur dioxide from about 0.1 to about 0.3%, preferably from 0.1 to about 0.3%) and steeping times of from about 0.1 to about 0.3%, from about 36 to about 48 hours, preferably from 36 to 48 hours at temperatures from about 45° C. to about 60° C., preferably from 45° C. to 60° C. The resulting steeped kernels are relatively softer than they were prior to steeping and at the end of the steeping process they can be separated into germs, fiber, starch and proteins.

The steeped grain is coarse ground in two steps to release the germ from the kernels. The germs are separated after each coarse milling step.

The remaining coarse de-germed kernels may be milled for a third time to disrupt the endosperm matrix and release the starch. Fibers are removed from the starch and endosperm proteins by passing the slurry over a series of screens.

The separated fiber is then dewatered and dried. At this point the main product stream contains starch, gluten and organic materials. The lower density gluten is separated from the starch by centrifugation, generally in two stages to obtain a high quality gluten, of 60 to 70% protein and 1.0 to 1.5% solids is then centrifuged to yield the supernatant of the corn gluten meal. This material can be concentrated by any known method.

The skilled person will appreciate that the description provided above is of an exemplary wet-milling process and that the process can be varied considerably from the above description. Therefore, it is understood that any method of separating the starch, protein, and fiber into separate streams can be used to provide the starting gluten enriched material needed for the disclosed uses and methods disclosed herein. The corn gluten concentrate used in the Examples of the current application typically has a dry matter content of approximately 13% and a protein concentration of approximately 60% on dry mass.

The process of producing a water soluble protein-containing aqueous fraction obtained from a wet-milling process can start using any water soluble protein-containing material that is produced during the wet-milling process.

Accordingly, the term “water soluble protein-containing aqueous fraction” refers to an aqueous fraction of any water soluble protein-containing stream that is generated in the wet-milling process (the aqueous fraction is that fraction of a wet milling stream that has been separated away from solid material, for example it may be a supernatant, and which thus may comprise soluble water components). The fraction may also be identified herein as the “aqueous fraction” or the “soluble fraction”

The protein in the water soluble protein-containing aqueous fraction obtained from a wet-milling process may comprise at least 10 molar % leucine (calculated on the basis of a combination of free amino acid and peptide bound amino acid), for example at least 11 molar %, for example at least 12 molar %, for example at least 15 molar %. In general less than 20 molar % leucine will be present.

Exemplary wet-mill streams which may be used to obtain the desired fraction include the fraction separated away from gluten meal, i.e., a gluten supernatant or filtrate. Accordingly, the preferred fraction for the uses described herein is a supernatant obtained from gluten meal, preferably from corn (maize) gluten meal.

The water soluble protein-containing aqueous fraction described above may be used in the applications described herein as is, preferably in a concentrated form, or, alternatively, may be enzymatically hydrolyzed prior to use. Accordingly, the water soluble protein-containing fraction described herein may be used in the form of a protein hydrolysate.

By protein hydrolysate, hydrolysate or hydrolysed protein is meant the product that is formed by enzymatic hydrolysis of the protein. A hydrolysate may be an enriched hydrolysate, i.e. a fraction of the protein hydrolysate for example enriched in selected peptides or wherein peptides or polypeptides have been removed from the hydrolysate. So a hydrolysate or an enriched hydrolysate is preferably a mixture of peptides (or a peptide mixture).

Such a peptide mixture may comprise a peptide population of which more than 50%, preferably even more than 60%, and most preferably more than 75% of the peptides present have a molecular weight below 400 Da. The implication of this is that the peptide mixture is relatively rich in di- and tripeptides. A protein hydrolysate suitable for use in the present invention may conveniently have a Degree of Hydrolysis (DH) of between 10 and 60, preferably a DH of between 20 and 55 or most preferably between 25 and 50. This DH value is monitored using a rapid OPA test (Nielsen, P. M., D. Petersen, and C. Dambmann. J. Food Sci. 2001; 66:642-646). The soluble fraction used as a starting point for the proteolytic hydrolysis according to the invention is hereby defined as having DH of 0.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. A protein is defined as used herein as the non-hydrolyzed protein. Moreover, protein can also mean hydrolyzed protein. By amino acid is generally meant free amino acid, which is thus not part of a peptide, polypeptide or protein.

According to a preferred embodiment of the process of the invention, the water soluble proteinaceous substrate described above is subjected to enzymatic hydrolysis. Typically, such hydrolysis may be carried out using an endoprotease and a tripeptidyl aminopeptidase (TPAP).

This enzymatic hydrolysis is preferably carried out using a serine protease (EC 3.4.21), a metallo endoprotease (EC 3.4.24) or an aspartic protease (EC 3.4.23). Optionally a combination of such endoproteases is used. Among the serine proteases the proline-specific endoprotease as obtained from A. niger (WO 02/45524) is of special interest for the present application. The class of metalloendoproteases can be separated into the neutral and acid metalloproteases. Well known examples of the neutral metalloproteases are bacillolysin (E.C. 3.4.24.28) and thermolysin (E.C. 3.4.24.27). Less well known examples of these neutral metalloendoproteases have been obtained from Aspergillus species. Unlike the serine and metalloendopeptidases, the aspartic proteases feature an acidic pH optimum that can be advantageously used in combination with the above mentioned proline-specific endoprotease and socalled tripeptidyl peptidases that also have acidic pH optima. The latter tripeptidyl peptidases are defined as enzymes capable of releasing tripeptides from a polypeptide. For the present application the socalled tripeptidyl-aminopeptidases (E.C. 3.4.14.9), releasing tripeptides from the N-terminal side of the polypeptide at acidic pH, are of special interest. Advantageously the protein substrate is first fermented with an endoprotease, such as a serine protease, metalloendoprotease or an aspartic protease, to partly hydrolyse the protein. We have found that the tripeptidylpeptidase is, in general, more effective on such prehydrolysed protein substrates and leads to high tripeptide levels in the final hydrolysate.

Tripeptidyl aminopeptidases are enzymes that can release tripeptides from the N-terminus of an oligopeptide. Tripeptidyl aminopeptidases (EC 3.4.14) have been isolated from mammalian as well as plant sources. Microorganisms from which tripeptidylpeptidases have been isolated are for example Streptomyces species (JP08308565, WO 95/17512 and U.S. Pat. No. 5,856,166)), Porphyromonas gingivalis (WO 00/52147), Dictyostelium discoidum and Aspergillus species (WO 96/14404). To date, the occurrence of tripeptidyl carboxypeptidases (EC 3.4.15) has been demonstrated in mammalian cells and in the microorganism Clostridium histolyticum only.

In all applications the protein hydrolysates according to the invention, offer attractive advantages such as lowered allergenicities, facilitated gastro-intestinal uptake, less chemical deterioration of desirable amino acids like glutamine and cystein and finally, absence of proteinaceous precipitations under acid conditions during prolonged storage periods. All these advantages can be combined if the hydrolysate is prepared using a combination of an endoprotease, preferably a proline specific endoprotease, and one or more tripeptidyl peptidases. According to the invention several useful tripeptidyl peptidases are preferably used in a pure or isolated state. Pure tripeptidyl peptidase can be obtained for example by overexpression of the enzyme in a suitable transformed host microorganism. Preferred are those tripeptidyl peptidases that exhibit a low selectivity towards the substrate to be cleaved, i.e. exhibit minimal amino acid residue cleavage preferences only. Combinations of tripeptidyl peptidases that hydrolyse high percentages of the naturally occurring peptide bonds are preferred. Despite this high activity to naturally occurring peptide bonds, a total hydrolysis to free amino acids is prevented by the nature of the tripeptidases. Also tripeptidyl peptidases that are optimally active between pH 4 to 8 and exhibit adequate temperature stability are preferred. Adequate temperature stability means that at least 40%, preferably at least 60%, more preferably between 70 and 100% of the initial hydrolytic activity survives after heating the enzyme together with the substrate for 1 hour at 50° C. Tripeptidyl aminopeptidase with an acidic pH optimum is the preferred tripeptidyl peptidase.

A mixture of tripeptidyl peptidases is especially preferred since such a mixture can decrease the reaction time. Moreover, a higher amount of tripeptides is formed if compared with the use of a single peptidase. Peptidases especially suitable in the present invention are described in WO 02/068623.

Accordingly, the hydrolysates generated from the water soluble protein-containing aqueous fraction described herein may be relatively rich in di- and tripeptides. Accordingly, the present invention provides a protein hydrolysate which is rich in one or more BCAAs, for example leucine-, and glutamine-containing dipeptide and/or tripeptides. The peptides may have a carboxy terminal proline. Preferably the protein hydrolysate of the invention is non-bitter. The hydrolysate may optionally comprise dipeptides.

A protein hydrolysate of the invention or a composition of the invention may comprise at least about 50 molar %, preferably at least 50 molar %, more preferably at least about 60 molar %, even more preferably at least 60%, still more preferably at least about 70 molar % or most preferably at least 70 molar % of the peptides have a molecular weight of 400 Da or less. At least about 20%, preferably at least 20%, more preferably at least about 25%, still more preferably at least 25%, even more preferably at least about 30% or most preferably at least 30% of the amino acids present in the peptides and free amino acids is a BCAA and/or glutamine whereby BCAA is leucine and/or isoleucine and/or valine.

In general less than about 60%, preferably less than 60%, more preferably less than about 50%, still more preferably less than 50% of the amino acids present in the peptides and free amino acids of the composition or hydrolysate of the invention is a BCAA and/or glutamine whereby BCAA is leucine and/or isoleucine and/or valine.

The process for generating such hydrolysates according to the invention involves a combination of one or more endoproteases with one or more tripeptidases (as described above). Advantageously, the enzymes may be used in an isolated form and in an endoprotease to tripeptidase protein weight ratio range between 1:0.05 and 1:50, preferably between 1:0.1 to 1:10.

The water soluble protein-containing aqueous fraction or a partial hydrolysate can first be subjected to the suitable first endoprotease and subsequently the TPAP or mixture of TPAP's can be added. In cases where the optimal activity conditions of the enzymes are roughly identical, a one step process may be preferred.

The present invention further provides a hydrolysate rich in tripeptides whereby preferably these tripeptides are rich in carboxy terminal proline. Rich in proline means that at least 20%, preferably at least 30%, more preferably at least 40% and even more preferably 50% of the proline present in the starting water-soluble protein substrate, is present in the tripeptides, preferably as carboxy terminal proline. Preferably 20% of the tripeptides or more preferably 35% of the tripeptides have a carboxy terminal proline residue.

The proteases suitable in the present invention are preferably recombinant and/or commercially available for food grade applications. Proteases produced using rDNA techniques, that is cloning the gene encoding the proteolytic activity in a host organism that over expresses this gene, usually provide for enzyme preparations comprising less contaminating enzymatic activities and thus may not require costly recovery steps. Well known host organisms that over express cloned genes include yeasts, fungi or bacteria (for example Saccharomyces, Kluyveromyces, Aspergillus, Trichoderma, E. coli, Bacillus etc.).

A protein hydrolysate or composition of the invention can be prepared by contacting the water-soluble protein fraction or substrate with one proteolytic enzyme or a combination of proteolytic enzymes. In the event that more than one protease is used, these proteases can be added to the protein substrate simultaneously. Alternatively, the proteases can be added to the protein in a predefined sequence. Optionally, the addition of the next protease is preceded by an inactivation of the protease or proteases that were used earlier in the hydrolysis process. Such inactivation may be achieved in various ways and the method of choice depends on the protease that has to be inactivated. Inactivation treatments include but are not limited to heat treatment and a change in pH.

The water-soluble protein-containing aqueous fraction or water-soluble composition according to the invention may be used, in non-hydrolysed or hydrolysed form (i.e. the hydrolysate of the invention), as is or may be subjected to additional processing before use, for example in order to obtain a protein concentrate in the form of, for example, a powder or paste. Accordingly, the soluble fraction, water-soluble composition or hydrolysate may e.g. be centrifuged and/or (micro or ultra) filtrated, then concentrated by e.g. evaporation or reversed osmosis, and optionally dried in any convenient way, such as spraydrying, freeze-drying, fluidised-bed treatment or vacuum drying. If the soluble fraction, water-soluble composition or hydrolysate are dried, the dry material may be ground and/or sieved in order to obtain fractions of a particular size range. Additional components may be added to the dried soluble fraction to facilitate drying or to influence the final characteristics of the dried fraction such as its tendency to form lumps or its wettability. For example, an anti-caking agent may be added.

A concentrated product will typically comprise at least about 20% dry matter (weight dry matter/total weight), preferably at least 20% dry matter (weight dry matter/total weight), more preferably at least about 30% dry matter (weight dry matter/total weight), still more preferably at least about 40% dry matter (weight dry matter/total weight, most preferably at least 40% dry matter (weight dry matter/total weight. A granulate or powder may comprise at least about 80% dry matter (weight dry matter/total weight), preferably at least 80% dry matter (weight dry matter/total weight), more preferably at least about 90% dry matter (weight dry matter/total weight), most preferably at least 90% dry matter (weight dry matter/total weight). Concentrated products preferably have a water activity (Aw) below about 0.85, preferably below 0.85, have an acidic pH value and are optionally preserved with a food grade preservative such as sorbate or benzoate.

The water soluble protein-containing aqueous fraction, water-soluble composition or hydrolysate described herein are suitable for a number of applications.

Examples of such applications include use in infant nutrition. Also, the aqueous fraction, water-soluble composition or hydrolysate may be used in products for consumers with non-medical needs such as individuals carrying out exercise. In this application, the aqueous fraction, water-soluble composition or hydrolysate may be used to aid or improve endurance and recovery during or after exercise. Thus, the aqueous fraction, water-soluble composition or hydrolysate may conveniently be incorporated into sports drinks. The aqueous fraction, water-soluble composition or hydrolysate may also be used, for example, in slimming products, products that help to improve body shape or products which make it easier to maintain body weight as well as products for raising or maintaining muscle mass. In this context, combining the consumption of the aqueous fraction, water-soluble composition or hydrolysate according to the invention in combination with exercise to increase the feeding induced stimulation of muscle protein synthesis is relevant. Accordingly, the aqueous fraction, water-soluble composition or hydrolysate may be used to stimulate the generation of lean body mass or maintain lean body mass. Typically, this will occur as a result of increasing or maintaining muscle mass, i.e. a gain or maintenance of muscle mass. By (muscle) protein gain is meant the net gain of the (muscle) protein which is the result of the protein breakdown of the protein in amino acids, the synthesis of protein, and the oxidation of amino acids. This application may be used by all people who need to maintain or increase their muscle mass, for example overweight people during a calorie restricted diet, children, women, the elderly and those who often skip meals. An increased protein intake combined with resistance training can stimulate muscle growth in people who are on a calorie restricted diet. Thus, increased muscle mass that burns more calories may help to improve body shape and make it easier to maintain body weight.

Apart from amino acids, carbohydrates are optionally present in the aqueous fraction, water-soluble composition or hydrolysate according to the invention. Depending upon the anticipated use, i.e. as such or in combination with other food, the aqueous fraction, water-soluble composition or hydrolysate may contain a separate source of carbohydrates. These carbohydrates can be glucose or more slowly absorbed carbohydrates like maltodextrins or starch depending upon the desired glycaemic-index for the particular application. In the composition of the invention, carbohydrates can be present in an amount of from about 1.0 to about 90% wt, preferably from 1.0 to 90% wt, more preferably from about 2 to about 50% wt, still more preferably from 2 to 50% wt, even more preferably from about 6 to about 35% wt, most preferably from 6 to 35% wt calculated on the basis of dry weight of the composition.

Of particular importance is the application of aqueous fraction, water-soluble composition or hydrolysate to treat certain medical conditions associated with inappropriate or abnormal blood sugar metabolism. Accordingly, the aqueous fraction, water-soluble composition or hydrolysate may be used for example in the treatment of type 1 or type 2 diabetes mellitus or for the prevention of diabetes in those individuals with pre-diabetes, or impaired glucose tolerance (IGT), metabolic syndrome, obesity as well as the application to prevent long-term complications in subjects with such disorders.

In another aspect, the present invention relates to the use of the aqueous fraction, water-soluble composition or hydrolysate as a nutritional supplement for the said treatment or prevention, e.g., as an additive to a multi-vitamin preparation comprising vitamins and minerals which are essential for the maintenance of normal metabolic function but are not synthesized in the body. In still another aspect, the invention relates to a method for the treatment of both type 1 and 2 diabetes mellitus and or obesity, or metabolic syndrome which comprises administering the soluble fraction or water-soluble composition to a subject in need of such treatment. Such nutritional supplements may also be used in the non-medical applications set out above.

The literature available indicates that especially the amino acid leucine plays an important role in stimulating insulin production in pre-diabetic individuals. Accordingly, the soluble fraction or water-soluble composition may be used in combination with additional free leucine and/or the soluble fraction or water-soluble composition may be hydrolysed in such a way so as to enrich the resulting hydrolysate specifically in free leucine as described in detail above and in WO02/032232.

Use according to the present invention may be useful in the long term treatment or prevention of type 2 diabetes or pre-diabetes or metabolic syndrome or obesity, or to prevent long-term complications in subjects with type 2 diabetes or pre-diabetes or metabolic syndrome or obesity. Preferably the present invention relates to the use of a composition comprising a protein hydrolysate as a nutraceutical, preferably a medicament, to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

The soluble fraction or water-soluble composition described herein or a processed form thereof may be incorporated into a food or feed product. Such a food or feed product may be a neutraceutical composition. The term neutraceutical in this context indicates the usefulness in both the nutritional and pharmaceutical field of application. Thus, the novel nutraceutical compositions can find use as supplement to food and beverages, and as pharmaceutical formulations for enteral or parenteral applications, which may be solid formulations such as capsules or tablets, or liquid formulations, such as solutions or suspensions. As will be evident from the foregoing, the term nutraceutical composition also comprises food and beverages containing a protein hydrolysate or a protein hydrolysate and leucine as well as supplement compositions containing the aforesaid active ingredients. Such food or feed products and food or feed supplements may be used in the applications described herein.

A multi-vitamin and mineral supplement may be added to the nutraceutical compositions of the present invention to obtain an adequate amount of an essential nutrients missing in some diets. The multi-vitamin and mineral supplement may also be useful for disease prevention and protection against nutritional losses and deficiencies due to lifestyle patterns and common inadequate dietary patterns sometimes observed in diabetes. Moreover, oxidant stress has been implicated in the development of insulin resistance. Reactive oxygen species may impair insulin stimulated glucose uptake by disturbing the insulin receptor signaling cascade. The control of oxidant stress with antioxidants such as alpha-tocopherol (vitamin E) ascorbic acid (vitamin C) may be of value in the treatment of diabetes. Therefore, the intake of a multi-vitamin supplement may be added to the above mentioned active substances to maintain a well balanced nutrition.

In a preferred aspect of the invention, the nutraceutical composition of the present invention contains the soluble fraction, water-soluble composition, the soluble fraction plus additional leucine or water-soluble composition plus additional leucine. Leucine suitably is present in the composition according to the invention in an amount to provide a daily dosage from about 0.001 g per kg body weight to about 1 g per kg body weight of the subject to which it is to be administered, preferably from 0.001 g per kg body weight to 1 g per kg body weight of the subject to which it is to be administered. A food or beverage suitably contains from about 0.05 g per serving to about 50 g per serving of leucine, preferably from 0.05 g per serving to 50 g per serving of leucine. If the nutraceutical composition is a pharmaceutical formulation such formulation may contain leucine in an amount from about 0.001 g to about 1 g per dosage unit, e.g., per capsule or tablet, preferably from 0.001 g to 1 g per dosage unit, e.g., per capsule or tablet, or from about 0.035 g per daily dose to about 70 g per daily dose of a liquid formulation, preferably from 0.035 g per daily dose to 70 g per daily dose of a liquid formulation. Protein hydrolysates suitably are present in the composition according to the invention in an amount to provide a daily dosage from about 0.01 g per kg body weight to about 3 g per kg body weight of the subject to which it is to be administered, preferably 0.01 g per kg body weight to 3 g per kg body weight of the subject to which it is to be administered. A food or beverage suitably contains from about 0.1 g per serving to about 100 g per serving of protein hydrolysates, preferably from 0.1 g per serving to 100 g per serving of protein hydrolysates. If the nutraceutical composition is a pharmaceutical formulation such formulation may contain protein hydrolysates in an amount from about 0.01 g to about 5 g per dosage unit, e.g., per capsule or tablet, preferably from 0.01 g to 5 g per dosage unit, e.g., per capsule or tablet, or from about 0.7 g per daily dose to about 210 g per daily dose of a liquid formulation, preferably from 0.7 g per daily dose to 210 g per daily dose of a liquid formulation.

Another important use of the soluble fraction or water-soluble composition described herein, especially when enriched specifically for leucine, is in increasing the glycogen level for a person in need of increased glycogen level or to rise the insulin secretion for a person in need thereof.

Such uses may be for example for athletes or other persons carrying out physical exercise, in particular to aid recovery and endurance. The soluble fraction or water-soluble composition may also be used as a source of amino acids to stimulate protein synthesis and thus have a positive influence on raising or maintaining muscle mass. The soluble fraction or water-soluble composition may thus be used by all people who need to maintain or increase their muscle mass, for example overweight people during a calorie restricted diet, children, women, the elderly and those who often skip meals. In combination with resistance training, intake of the soluble fraction or water-soluble composition can stimulate muscle growth in people who are on a calorie restricted diet and increased muscle mass that burns more calories may help to improve body shape and make it easier to maintain body weight. Obviously the soluble fraction or water-soluble composition according to the invention is not only beneficial for human application but is also advantageously used in other mammals such as pets, racing dogs and horses.

The soluble fraction or water-soluble composition may suitably be used as an additive for use in any energy supplementation or metabolic nutrient. The energy supplementation or nutrient can be in the form of beverage, such as sports drinks, energy drinks or other soft drinks, or any other nutrient preparation suitable for an athlete or another person in need of increased glycogen level or increased insulin production. The energy supplementation or nutrient is preferably in a form that it can be orally consumed. This increasing the glycogen level or the rise of the insulin secretion may for example lead to faster rebuilding of glycogen depots and faster rebuilding of degraded muscular proteins.

A sports drink is a beverage which is intended for rehydration, for example rehydration of an athlete, as well as restoring electrolytes, sugar and other nutrients. Sports drinks are usually isotonic, meaning they contain the same proportions of nutrients as found in the human body.

Energy drinks are beverages which contain (legal) stimulants, vitamins (especially B vitamins) and minerals with the intent to give the user a burst of energy. Common ingredients include caffeine, gurana (caffeine from the Guarana plant), taurine, various forms of ginseng, maltodextrin, inositol, carnitine, creatine, glucoronolactone and ginko biloba. Some may contain high levels of sugar, for example sucrose and/or glucose. Such beverages may be flavored and/or colored.

A soft drink is a drink that does not contain alcohol, as opposed to a hard drink that does. In general, the term is used only for cold beverages since hot chocolate, tea and coffee are not typically considered as soft drinks. The term originally referred exclusively to carbonated drinks and is still commonly used in this manner.

All such beverages mentioned above may comprise the soluble fraction or water-soluble composition as described above.

The soluble fraction or water-soluble composition may be used in combination with an insulin sensitizer. This is described in detail in WO04/022083. By ‘insulin sensitizing agent’ or ‘insulin sensitizer’ is meant a compound that will lower blood glucose levels by increasing the responsiveness of the tissues to insulin. Examples of “natural” insulin sensitizing agents are minerals preferably chromium, vanadium or a B-vitamins like niacin. Furthermore herbs or plant extracts preferably from banaba leaf, ginseng berry, cinnamon and certain compounds in grapes have been shown to be effective insulin sensitizers. Also the active compounds identified in these herbs and plant extracts and thought to be responsible for these insulin sensitising effects are preferably applied as natural sensitizers: corosolic acid, pterostilbene, methylhydroxy chalcone polymer (MHCP) and Ginsensoside Re. Preferred examples of pharmaceutical “insulin sensitizers” are biguanides (such as Metformin (e.g. Glucophage®) and thiazolidinediones (such as Pioglitazone (e.g. Actos®) and Rosiglitazone (e.g. Avandia®)). These natural insulin sensitizers be preferably added to products in quantities according to their “Reference Daily Intake” or even higher, depending on the person's nutritional need. A list for Reference Daily Intake values is included in the FDA's Code of Federal Regulations 21 CF 101.9, Apr. 1, 2001 which includes those nutrients for which a RDI has been established [http://vm.cfsan.fda.gov/˜Ird/CFR101-9.HTML].

The composition according the invention may contain a single insulin sensitizing agent or combinations of such agents. Preferably the composition according to the invention contains these agents in their recommended daily dosages or higher. The composition according to the invention may contain the peptide fraction and the insulin sensitizer in a mixed form or the peptide fraction and insulin sensitizer may be a separately packed and sold as a one package. Also the use of the composition according to the invention in combination with a pharmaceutical insulin sensitiser may be recommended.

The soluble fraction or water-soluble composition described herein may also be used to modulate flavour in a food or feed product (when it is incorporated into the said food or feed product during its preparation). Such a soluble fraction or water-soluble composition may be used in the form of a hydrolysate, in particular one on which the amount of one or more amino acids is specifically enriched.

A soluble fraction or water-soluble composition for use according to the invention enriched in a specific amino acid such as leucine or set of amino acids which include leucine can be used to impart a specific flavour profile on a food or food ingredient. This specific flavour profile can be generated during a fermentation process or during heating in a Maillard type of reaction.

Although many amino acids have been indicated in the aroma development of fermented products like cheese, bread, fermented sausages and beers, hydrophobic amino acids like the BCAA's leucine, valine and isoleucine are known to be of particular importance. Therefore, the soluble fraction or water-soluble composition for use according to the invention, optionally one obtained after protein hydrolysis, may be directly used in improving the aroma and taste profile of such fermented food products. In the preparation of a fermented flavour, the aqueous suspension comprising the protein hydrolysate e.g. after concentration, may be incubated with a food grade microorganism, under conditions suitable for the microorganism to ferment the protein hydrolysate. After fermentation, a fermented flavour may be obtained, e.g. by concentrating and/or drying of the fermented product. Therefore, in another embodiment of the application, the soluble fraction or water-soluble composition for use according to the invention may be used in the preparation of fermented food ingredients or products as described above. Fermented food ingredients or products are prepared by at least one fermentation step, wherein either enzymes endogenous to the food treated are actively involved or food grade microorganisms are incubated with a foodstuff to obtain the fermented foodstuff. Examples of fermented foodstuffs are for instance meat products such as hams or sausages or levened bread or yogurt, cheese, beer, whiskey, wine and champagne. Fermented food flavours, are flavours obtained from a fermented foodstuff. These fermented food flavours can be obtained by incubating the soluble fraction or water-soluble composition for use according to the invention with a food grade microorganism, which will ferment the soluble to desirable food flavours.

A process for producing a fermented food product, wherein a soluble fraction or water-soluble composition for use according to the invention has been used, will involve the addition of the protein hydrolysate before, or during the fermentation of that fermented food product. The product's endogenous enzymes or the fermenting organisms will convert the specific amino acid in additional flavours characteristic for this type of fermented food. Also flavours not-characteristic for this type of fermented food products may be produced in this way, resulting in fermented food products with surprising new flavours.

The fermented food product may be beer and the soluble fraction or water-soluble composition may be added to the vessel where the fermentation by the brewer's yeast takes place. The fermented food product may be bread and the soluble fraction or water-soluble composition may be added together with the flour to obtain a dough to which bakers yeast is added.

In yet another application of the inventive product, the soluble fraction or water-soluble composition for use according to the invention may be used in the preparation of reacted flavours or to enhance Maillard reactions during the cooking, baking or roasting phase of the food preparation as occurs for example, during the baking of bread especially on the crust (wherein a protein hydrolysate of the invention has been added to the dough or on top of the dough). In both the preparation of reacted flavors and in food preparations in which aroma and taste is enhanced via a Maillard reaction, the free amino acid content of the soluble fraction or water-soluble composition is preferably enhanced using an exoprotease, optionally in combination with an endoprotease and/or a tripeptidyl peptidase. Such an exoprotease is preferably a carboxypeptidase, more preferably a carboxypeptidase from A. niger and capable of releasing free amino acids. Optionally, the protein hydrolysate and an appropriate sugar can also be pre-reacted (in the absence of the other food ingredients, for example with a selected sugar) to obtain a suitable flavouring ingredient.

The soluble fraction or water-soluble composition and a sugar comprising composition are then reacted to obtain the reacted flavour composition. Sugars may e.g. be selected from aldoses and ketoses as well as disaccharides such as xylose, xylulose, glucose, fructose, maltose, sucrose or mixtures thereof.

The soluble fraction or water-soluble composition for use according to the invention and at least one reducing sugar are heated to start a series of reactions known as the Maillard reactions. Amino groups (particularly of free amino acids) react with reducing compounds as a first step. A whole family of other reaction pathways will follow, and finally results in a (complex) reacted flavour composition. By the use of the soluble fraction or water-soluble composition for use according to the invention, novel types of flavours can be obtained. These (reacted) flavours can be added to foodstuffs to improve the flavour of foodstuffs. To prepare the reacted flavour the amino acid enriched soluble and the desired sugar are dissolved in water in an appropriate ratio and then heated. Upon the dissipation of all water the heating process may be stopped immediately or may be continued to reach temperatures of around 120° C. or even 180° C. The latter incubation conditions lead to vastly different flavour and aroma profiles. Ultimately the dry, reacted product may be recovered as a powder and used as a flavouring ingredient.

Alternatively the soluble fraction or water-soluble composition for use according to the invention and sugar may be reacted in mixtures containing water and e.g. oil or fat, or in the total absence of water e.g. by dissolving sugar and soluble fraction or water-soluble composition in an essentially water-free system such as for example a polyalcohol. Advantage of this last approach is that even at temperatures above 100° C. the reaction takes place in a liquid and also the final product is a liquid which facilitates the dosing of the flavouring ingredient.

The following Examples illustrate the invention:

### EXAMPLES

### Materials & Methods

Unless indicated otherwise, all enzymes used were obtained from DSM Food Specialties (Delft, The Netherlands).

Mucorpepsin (Fromase XLG 750 IMCU/ml) was used at a dosage of 45 International Milk Clotting Units (IMCU)/g protein present (ref.: IDF (1997). Bovine rennets. Determination of total milk-clotting activity. International IDF Standard 157A. International Dairy Federation, Brussels, Belgium).

The proline specific endoprotease from Aspergillus niger was overproduced as described in WO 02/45524. The activity of the enzyme was tested on the synthetic peptide Z-Gly-Pro-pNA at 37° C. in a citrate/disodium phosphate buffer pH 4.6. The reaction product was monitored spectrophotometrically at 405 nm. One unit (PPU) is defined as the quantity of enzyme that liberates 1 μmol of p-nitroanilide per minute under these test conditions.

The tripeptidyl aminopeptidase from A. niger (sequence number 12 of WO02/068623) was overproduced as described for the proline-specific endoprotease. Its activity (80 units/a) was measured using the synthetic substrate Ala-Ala-Phe-pNA (Bachem, Switzerland) in an incubation in 0.1 mol/litre citrate buffer at pH 4.0 and 60° C. One unit is defined as the quantity of enzyme that provokes the release of 1 μmol of p-nitroanilide per minute under these conditions.

Also CPD-1 (Pep-G) was overproduced in A. niger. The activity of this enzyme was measured using the synthetic substrate FA-Phe-Ala (Bachem, Switzerland) in an an incubation with 1.5 mM FA-Phe-Ala, pH 4.5 at 37° C. One enzymatic unit of CarboxyPeptidase G (CPGU) is the amount of enzyme needed to decrease the optical density at 340 nm by one absorbency unit per minute (1 AU/min) under the conditions of the test.

The subtilisin used was “Protex 6L” from Genencor (Leiden, The Netherlands).

### Amino Acid Analyses

Amino acid analyses were carried out according to the PicoTag method as specified in the operator's manual of the Amino Acid Analysis System of Waters (Milford Mass., USA). To that end, samples were dried and directly derivatised using phenylisothiocyanate. The derivatised amino acids present were quantitated using HPLC methods as described. As during the usual acid hydrolysis Trp and Cys are destroyed, special methods are required to quantitate these two amino acids. In the present amino acid analyses, these amino acids Trp and Cys were not determined and are not included in the relevant Tables.

### Degree of Hydrolysis

The Degree of Hydrolysis (DH) as obtained during incubation with the various protolytic mixtures was monitored using a rapid OPA test (Nielsen, P. M.; Petersen, D.; Dambmann, C. Improved method for determining food protein degree of hydrolysis. Journal of Food Science 2001, 66, 642-646). The soluble fraction used as a starting point for the proteolytic hydrolysis according to the invention is hereby defined as having DH of 0.

### Kjeldahl Nitrogen

Total Kjeldahl nitrogen was measured by Flow Injection Analysis. Using a Tecator FIASTAR 5000 Flow Injection System equipped with a TKN Method Cassette 5000-040, a Pentium 4 computer with SOFIA software and a Tecator 5027 Autosampler the ammonia released from protein containing solutions was quantitated at 590 nm. A sample amount corresponding with the dynamic range of the method (0.5-20 mg N/I) was placed in the digestion tube together with 95-97% sulphuric acid and a Kjeltab subjected to a digestion program of 30 minutes at 200° C. followed by 90 minutes at 360 degrees C. After injection in the FIASTAR 5000 system the nitrogen peak is measured from which the amount of protein measured can be inferred.

### Molecular Weight Distribution of Peptides

Analysis of the peptide size distribution was carried out on an automated HPLC system equipped with a high pressure pump, an injection device able to inject 10-100 microliter sample and a UV detector able to monitor the column effluent at 214 nm, a wavelength deliberately chosen because it is a measure for peptide bonds. Analysis was carried out on a Superdex Peptide HR 10/300 GL column (Amersham) equilibrated with 20 mM sodium phosphate/250 mM sodium chloride pH 7.0 buffer. After injecting a sample (typically 50 microliter) the various components were eluted from the column with buffer in 90 min at a flow rate of 0.5 ml/min. The system was calibrated using a mixture of cytochrome C (Mw 13 500 Da), aprotinin (Mw 6510 Da) and tetra-glycine (Mw 246 Da) as molecular weight markers.

### Example 1

### Dissolving Suspended Maize Gluten Protein Using Enzymes is Surprisingly Ineffective

Corn gluten meal is a major co-product of corn wet milling. The product is relatively cheap, rich in protein and features high BCAA, such as leucine, and glutamine concentrations. Unfortunately, the proteins present in the product have a low water solubility. To improve the latter aspect, we tested the effect of a number of different proteases.

Corn gluten meal samples were obtained as suspensions from a local corn processing plant. Maximal susceptibility to proteolytic attack was guaranteed by minimizing protein denaturation. To that end, the sample material was obtained and kept as frozen suspensions rather than spray-dried material. In view of the pH 3.9 of the corn gluten meal suspension obtained and the fact that corn glutens are maximally soluble at such acid pH values, the use of proteases with acid pH optima was judged to be most efficacious. On the basis of its published amino acid composition, proteolytic endoproteases with an acid pH optimum and preferring cleavage at the ubiquitous amino acids proline and leucine were seen as most appropriate for hydrolysis. Mucorpepsin (EC 3.4.23.23) is a well known industrial enzyme preferring cleavage of peptide bonds involving leucine. The proline-specific endoprotease from A. niger is a unique enzyme that can cleave the C-terminal of proline residues at acidic pH values (WO 02/45524). Therefore, incubation of the corn gluten suspension with these two endoproteases can be expected to solubilise a large variety of different gluten peptides hereby increasing the nitrogen content of the aqueous phase. For an even greater degradation of these solubilised peptides, exoproteases releasing free amino acids can be used. In the present experiment, we tested such an exoprotease option by using an acid serine-type carboxypeptidase (EC 3.4.16.1), more specifically CPD-1 (Pep-G) of A. niger (Dal Degan et al., 1992. Appl. Environ. Microbiol. 58(7) 2144-2152).

The corn gluten meal starting material used had a dry matter content of 12.9% and a protein content of 8.5% (Kjeldahl factor 6.25). The enzymes used were either mucorpepsin alone (Fromase XLG 750, amylase free, at a dosage of 45 IMCU/g protein), a combination of mucorpepsin plus the proline-specific endoprotease (from A. niger at a dosage of 4 PPU/g protein) or a combination of mucorpepsin plus proline-specific protease plus carboxypeptidase (CPD-1 at a dosage of 260 CPGU/g protein). To maximize cleavage and thus dissolution, the various enzyme incubations were carried out simultaneously for 24 hours at a temperature of 45° C. After incubation, the various samples were centrifuged and the protein content in the supernatant fractions was determined. Although all enzymes were found to increase the level of dissolved protein, the proline-specific endoprotease and the exo-protease PepG had the biggest impact (see Table 1). However, even in combination, the three enzymes could dissolve no more than 50% of the total protein present. In view of the relatively low cost of the corn gluten meal and the appreciable costs of the high enzyme concentrations used, the enzymatic hydrolysis turned out to be not cost-effective.

TABLE 1
Increase of water-soluble nitrogen after incubation
with various enzyme combinations.

| | Delta Kjeldahl | Percentage water soluble |
|---|---|---|
| Enzyme | nitrogen | protein of total protein present |
| Mucorpepsin | 0.5 | 14 |
| Proline-specific | 2.5 | 32 |
| protease | | |
| Carboxypeptidase | 1.5-2.0 | 46 |

### Example 2

### Amino Acid Compositions of Corn Gluten Meal Suspensions and Supernatants

According to a number of recent scientific publications, the consumption of specific amino acid compositions can have physiological and even psychological effects. Furthermore, adding specific amino acid compositions to fermentation processes affects the taste and aroma of the final product. To characterize the suitability of the corn gluten meal for specific dietary applications, we evaluated the amino acid compositions of the corn gluten meal suspension and its water soluble supernatant fraction. Amino acid compositions obtained with acid hydrolysis of the proteinaceous material were determined as specified in the Materials & Methods section. The data obtained after acid hydrolysis are shown in Table 2. According to the data obtained, in both the total suspension and in the supernatant, relatively high levels of leucine, BCAA's and glutamine are present. The level of free plus peptide bound amino acids present in the supernant was found to be about twice as high as its level of free amino acids.

Noteworthy is that the level of free leucine present in the supernatant is high (about 17% of all free amino acids present) and the level of free glutamine is low (about 3% of all free amino acids present). Please note that the latter data on the levels of free leucine and glutamine present were measured using an amino acid analysis without an acid hydrolysis step. As described earlier, such an amino acid composition in combination with the relatively low costs of the products involved (see Example 3), make the various fractions of the corn gluten supernatant particularly suited for dietary intervention.

TABLE 2
Total amino acid composition of corn gluten meal and
its supernatant as obtained after acid hydrolysis

| | | Total suspension | | Supernatant | |
|---|---|---|---|---|---|
| | Amino acid | Micromol/g | % | Micromol/g | % |
| Large | Leu | 167.6 | 15.9 | 12.8 | 10.9 |
| hydrophobic | | | | | |
| | Ile | 41.4 | 3.9 | 4.2 | 3.6 |
| | Val | 55.3 | 5.2 | 7.0 | 5.9 |
| | Phe | 53.0 | 5.0 | 4.0 | 3.4 |
| | Tyr | 30.9 | 2.9 | 1.8 | 1.5 |
| | Pro | 114.7 | 10.9 | 15.4 | 13.1 |
| Basic | Arg | 25.8 | 2.4 | 2.8 | 2.4 |
| | Lys | 14.5 | 1.4 | 3.7 | 3.1 |
| | His | 20.2 | 1.9 | 3.1 | 2.6 |
| Acidic | Glu | 175.2 | 16.6 | 16.0 | 13.6 |
| | Asp | 50.7 | 4.8 | 6.8 | 5.8 |
| Small | Gly | 54.4 | 5.1 | 10.4 | 8.8 |
| neutral | Ala | 126.7 | 12.0 | 15.6 | 13.2 |
| | Ser | 68.0 | 6.4 | 7.1 | 6.0 |
| | Thr | 38.9 | 3.7 | 5.0 | 4.2 |
| S-containing | Met | 19.8 | 1.9 | 2.2 | 1.9 |
| Total | | 1057.1 | 100.0 | 117.9 | 100.0 |

Trp and Cys were omitted from this analysis; during the amino acid analysis procedure, the glutamine present is converted into glutamate.

### Example 3

### Recovery of Maize Gluten Protein from the Soluble Fraction is Cost-Effective

The results obtained in Example 1 indicate that the option to solubilise the water insoluble fraction of maize gluten using proteolytic enzymes is economically not attractive. As an alternative, we calculated the costs of concentrating the water soluble protein fraction as present in the supernatant of the corn gluten meal suspension. To that end a process was conceived in which the corn gluten meal was decanted and the supernatant, optionally after a filtration step, was evaporated to a dry matter content of approximately 500 g/kg. The resulting concentrate was used as such or spray-dried to obtain a dry powder. The cost of the latter concentrated or spray-dried protein fraction was surprisingly low. In combination with its attractive amino acid composition (see Example 2), these low costs make the corn gluten supernatant a very attractive substrate for various food or feed applications.

### Example 4

### The Soluble Fraction of Maize Gluten is Ideally Suited for Dietary Applications

During the past few years, the benefits of protein hydrolysates with enhanced levels of readily absorbable leucine, glutamine and BCAA's are being elucidated. As illustrated in Example 2, the corn gluten supernatant is quite rich in these specific amino acids. Among the proteins widely available for industrial application, only whey protein is comparable in terms of amino composition but this protein is relatively expensive, especially if enzymatically hydrolysed.

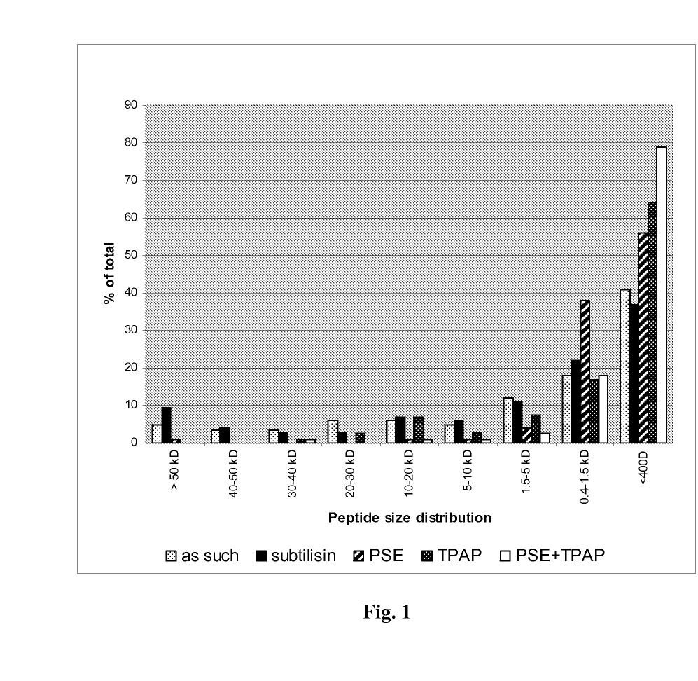

Ideally, the leucine, glutamine and BCAA's as present in the corn gluten supernatant are presented in their most effective form, that is, readily available for transport over the intestinal wall into the bloodstream. Only if presented in the form of either a free amino acid or incorporated in a di- or a tri-peptide, absorption into the blood is swift leading to surging amino acid levels. As already mentioned in Example 2, about one half of all amino acids present in the corn gluten supernatant, is present as the free amino acid. The other half is present in the form of peptides of different sizes. To get an impression of their molecular weights, the size distribution of the peptides present in the corn gluten supernatant was analyzed. As can be seen in FIG. 1, only 40% of all peptides present in the corn gluten supernatant, has the preferred size of a tri- or di-peptide (Mw<400 Da). To increase the portion of these tri- and di-peptides in the supernatant, we treated the supernatant with various proteolytic enzymes. Note that in this application only moderate enzyme concentrations are required as the larger part of the corn gluten, i.e. the non-soluble corn glutens, are no longer present in the supernatant. In contrast with the situation described in Example 1, such moderate enzyme concentrations can be applied cost-effectively. As can be seen in FIG. 1, treatment with the widely used and cost-effective endoprotease subtilisin (Protex, Genencor) has no effect on the level of di- and tri-peptides present. Presumably because this enzyme has a near neutral pH optimum. However, treatment with the proline-specific protease from A. niger (4 PPU/g protein present) and, especially treatment with a combination of the proline-specific protease and a so-called tripeptidyl amino peptidase (see Materials & Methods; 12 units/gram protein present), generates an end product in which 80% of all peptides are present in the form of a readily absorbable di- or tripeptides. Therefore, to ensure optimal bio-activity, the peptides present in the corn gluten supernatant are preferably treated with suitable proteases to generate a high proportion of di- and tri-peptides.

1. A water-soluble composition comprising free amino acids and peptides whereby the weight ratio of free amino acids to peptides is between 3:1 and 1:3, preferably between 2:1 and 1:2 and wherein at least about 50 molar % of the peptides has a molecular weight of 400 Da or less.

2. A water-soluble composition according to claim 1 wherein at least about 20%, preferably at least 20%, more preferably at least about 25%, still more preferably at least 25%, even more preferably at least about 30% or most preferably at least 30% of the amino acids present in the peptides and free amino acids is a BCAA and/or glutamine whereby BCAA is leucine and/or isoleucine and/or valine.

3. A water-soluble composition according to claim 1 wherein at least 50 molar %, more preferably at least about 60 molar %, still more preferably at least 60 molar %, even still more preferably at least about 70 molar %, or most preferably at least 70 molar % of the peptides have a molecular weight of 400 Da or less.

4. A water-soluble composition according to claim 1, wherein the peptides are rich in di- or tripeptides.

5. A water-soluble composition according to claim 1, wherein the composition has a vegetable origin and is preferably the hydrolysate of a vegetable protein or vegetable protein fraction.

6. A water-soluble composition according to claim 1 which is obtainable by the enzymatic protein hydrolysis of a water soluble protein fraction from a corn wet-milling process.

7. A method for preparing a protein hydrolysate, the method comprising contacting a water soluble protein-containing aqueous fraction obtained from a protein wet-milling process with:
a) an endoprotease; and
b) an exoprotease preferably a tripeptidase, more preferably a tripeptidylaminopeptidase(TPAP).

8. A method according to claim 7 wherein the endoprotease is a proline specific endoprotease (PSE), a serine protease, an aspartic protease or a metalloendoprotease.

9. A protein hydrolysate obtainable by a method according to claim 7 preferably wherein at least about 50 molar %, preferably at least 50 molar %, more preferably at least about 60 molar %, still more preferably at least 60 molar %, even still more preferably at least about 70 molar %, or most preferably at least 70 molar % of the peptides have a molecular weight of 400 Da or less.

10. Use of a water soluble protein-containing aqueous fraction obtained from a corn wet-milling process or a water-soluble composition according to claim 1 in:
the manufacture of a medicament for the treatment or prevention of a condition associated with inappropriate blood sugar metabolism;
aiding recovery and/or endurance during or after exercise;
stimulating the generation of lean body mass;
reducing diet-induced obesity;
improving cholesterol metabolism;
overcoming liver failure and cirrhosis;
infant nutrition;
the preparation of a food or feed composition or a food or feed supplement; or
modulating the taste or aroma of fermented or heated food products.

11. Use according to claim 10, wherein the water soluble protein-containing aqueous fraction obtained from a wet-milling process is obtained from a corn wet-milling process or is the supernatant fraction of corn gluten meal.

12. Use according to claim 10, wherein the protein in the water soluble protein-containing aqueous fraction obtained from a wet-milling process has been subjected to hydrolysis, preferably enzymatic hydrolysis.

13. Use according to claim 10 wherein the water soluble protein-containing aqueous fraction obtained from a wet-milling process comprises at least about 20%, preferably at least 20%, more preferably at least about 25%, still more preferably at least 25%, even more preferably at least about 30% or most preferably at least 30% of the amino acids present in the peptides and free amino acids is a BCAA and/or glutamine whereby BCAA is leucine and/or isoleucine and/or valine

14. Use according to claim 10 wherein the soluble fraction of a protein-containing material obtained from a wet-milling process comprises carbohydrate.

15. Use according to claim 10, wherein the condition associated with inappropriate blood sugar metabolism is diabetes, pre-diabetes, impaired glucose tolerance, metabolic syndrome or obesity, preferably wherein the diabetes is type 1 or type 2 diabetes.

16. Use according to claim 10:
for the long term treatment or prevention of type 2 diabetes or prevention of diabetes in an individual with pre-diabetes, metabolic syndrome or obesity;
to prevent long-term complications in an individual with type 2 diabetes, pre-diabetes, metabolic syndrome or obesity;
to decrease 24-hour blood glucose levels in an individual with type 2 diabetes, pre-diabetes, metabolic syndrome or obesity;
to increase 24-hour insulin secretion in an individual with type 2 diabetes, pre-diabetes, metabolic syndrome or obesity;
to decrease glycosylated hemoglobin concentration (HbAlc) in an individual with type 2 diabetes, pre-diabetes, metabolic syndrome or obesity;
to reduce the length of hyperglycaemic periods in an individual with type 2 diabetes, pre-diabetes, metabolic syndrome or obesity; or
to reduce mortality in an individual with type 2 diabetes or pre-diabetes or metabolic syndrome or obesity.

17. Use according to claim 10, wherein stimulating the generation of lean body mass is a result of increased generation of muscle mass.

18. Use according to claim 10, wherein the water soluble protein-containing aqueous fraction obtained from a wet-milling process obtained from a wet-milling process is capable of improving the flavour or aroma of a food or feed composition or of a food or feed supplement, preferably wherein the food composition is a fermented food of feed composition.

19. A method for the preparation of a food or feed composition or a food or feed supplement which method comprises use of a composition according to claim 1 or a water soluble protein-containing aqueous fraction obtained from a wet-milling process during preparation of a said food or feed composition.

20. A method according to claim 18, wherein the water soluble protein-containing aqueous fraction obtained from a wet-milling process is as defined in claim 10.

21. A food or feed composition or a food or feed supplement prepared using a composition according to claim 1 or a hydrolysate or a water soluble protein-containing aqueous fraction obtained from a wet-milling process, preferably wherein the food or feed composition is a fermented composition.

22. A water soluble protein-containing aqueous fraction obtained from a wet-milling process or a composition according to claim 1 or a hydrolysate for use in the treatment or prevention of a condition associated with inappropriate blood sugar metabolism.

23. A method for the treatment or prevention of a condition associated with inappropriate blood sugar metabolism, which method comprises administering to an individual in need thereof a therapeutically effective amount of a water soluble protein-containing aqueous fraction obtained from a wet-milling process or a composition according to claim 1 or a hydrolysate.
